(19) United States
(12) Patent Application Publication
(10) Pub. No.: US 2015/0051440 A1
(43) Pub. Date: Feb. 19, 2015

(54) METHOD OF CALMING AN INFANT USING AN ATTACHABLE VIBRATORY DEVICE

(71) Applicant: Loraine Buck, Fleming Island, FL (US)

(72) Inventor: Loraine Buck, Fleming Island, FL (US)

(21) Appl. No.: 14/459,123

(22) Filed: Aug. 13, 2014

Related U.S. Application Data
(60) Provisional application No. 61/865,332, filed on Aug. 13, 2013.

(51) Int. Cl. A61M 21/02

(52) U.S. Cl. CPC A61M 21/02; A61M 2021/0022
USPC 600/27

(57) ABSTRACT

A method of utilizing an infant calming attachable vibratory device which is structured for attachment to various infant devices, such as for example strollers, carriers, car seats, cribs, etc., wherein the vibratory device produces steady or rhythmic vibrations that are of sufficient strength to transfer to the infant device and be felt by an infant retained within the infant device. In a preferred embodiment, the vibratory device is a battery powered assembly having a housing containing an arm to which is mounted a weight, the arm pivoting or rotating about an axis, wherein the rotation of the weight imparts a vibration or wobble to the housing. In a more preferred embodiment, the rate and/or amount of wobble is adjustable by changing the weight to a lighter or heavier member. The vibratory device is constructed such that the vibration or wobble rate per minute is within the typical range of heartbeats per minute of an expectant mother, from approximately 75-95 beats per minute.

This application claims the benefit of U.S. Provisional Patent Application Ser. No. 61/865,332, filed on Aug. 13, 2013, the disclosure of which is incorporated herein by reference.

### BACKGROUND OF THE INVENTION

The invention relates generally to the field of vibratory devices, and in particular relates to the field of vibratory devices that are structured and designed for attachment to infant accessories, carriers, cribs or the like, whereby the vibrations produced by the device transfer to the accessory, carrier, crib or like infant device in order to calm or soothe an infant retained by or associated with the infant device.

It is well known that steady or rhythmic vibrations, oscillations, rocking or the like of infants have a calming effect on the infants, often inducing sleep. For example, the use of rocking chairs, swinging cribs or even rides in an automobile are known techniques for getting infants to go to sleep. To this end, it is known to provide vibratory devices for attachment to infant devices, such as strollers, carriers, car seats, cribs, etc. Such devices can be seen, for example, in U.S. Pat. No. 5,660,597, U.S. Pat. No. 5,074,820, U.S. Pat. No. 4,785,797, U.S. Pat. No. 3,311,935, and U.S. Pat. No. 2,932,821, the disclosures of which are incorporated herein by reference.

It is an object of this invention to provide a method of calming an infant using an attachable vibratory device that improves on the known devices by providing an oscillating or rhythmic vibration pattern equivalent to a maternal heartbeat rate.

### SUMMARY OF THE INVENTION

The invention is a method of utilizing an infant calming attachable vibratory device which is structured for attachment to various infant devices, such as for example strollers, carriers, car seats, cribs, etc., wherein the vibratory device produces steady or rhythmic vibrations that are of sufficient strength to transfer to the infant device and be felt by an infant retained within the infant device. In a preferred embodiment, the vibratory device is a battery powered assembly having a housing containing an arm to which is mounted a weight, the arm pivoting or rotating about an axis, wherein the rotation of the weight imparts a vibration or wobble to the housing. In a more preferred embodiment, the rate and/or amount of wobble is adjustable by changing the weight to a lighter or heavier member. In another preferred embodiment, the vibratory device is constructed such that the vibration, oscillation or wobble rate per minute is within the typical range of heartbeats per minute of an expectant mother, approximately 80-90 beats per minute in a majority range, and 75-95 when almost all normal rates are included. The device is attached to the infant device containing the infant and activated such that the vibration, oscillation or wobble occurs at approximately 80 to 90 times per minute.

### BRIEF DESCRIPTION OF THE DRAWINGS

FIG. 1 is an exploded view of a representative infant calming vibratory device.

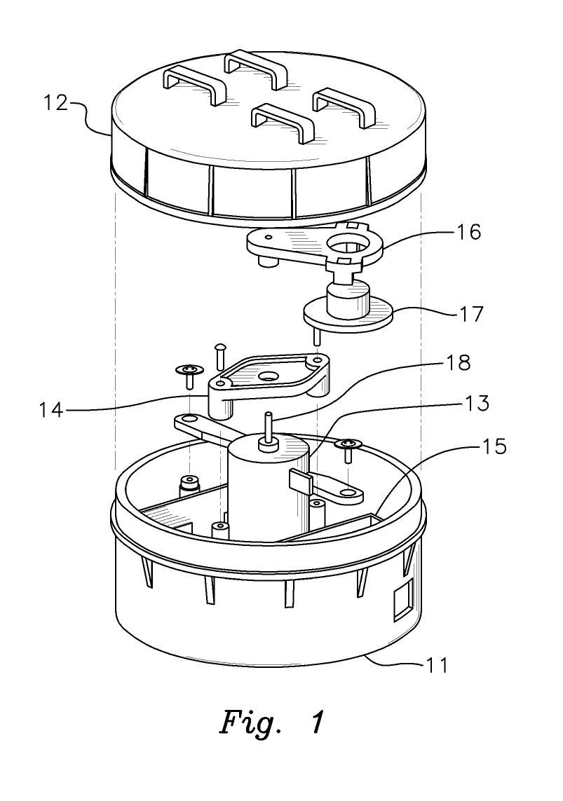

FIG. 2 illustrates a representative infant calming device with an attachment strap.

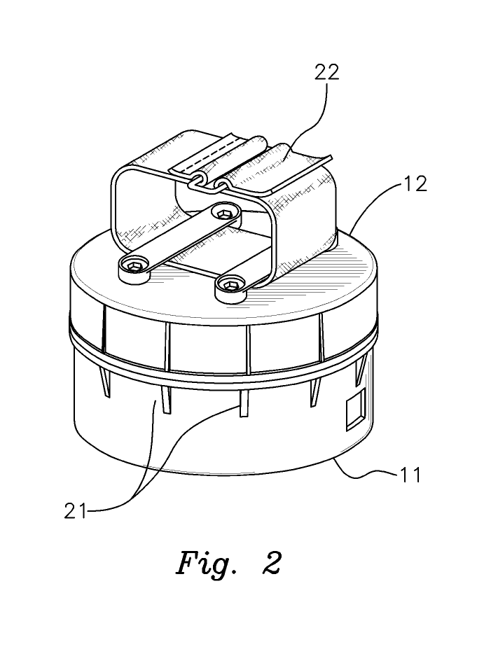

### DETAILED DESCRIPTION OF THE INVENTION

The invention is a method of using an infant calming attachable vibratory device which is structured for attachment to various infant devices, such as for example strollers, carriers, car seats, cribs, etc., wherein the vibratory device produces steady or rhythmic vibrations that are of sufficient strength to transfer to the infant device and be felt by an infant retained within the infant device. In a preferred embodiment, the vibratory device is a battery powered assembly having a housing containing an arm to which is mounted a weight, the arm pivoting or rotating about an axis, wherein the rotation of the weight imparts a vibration or wobble to the housing. In a more preferred embodiment, the rate and/or amount of wobble is adjustable by changing the weight to a lighter or heavier member. In another preferred embodiment, the vibratory device is constructed such that the vibration, oscillation or wobble rate per minute is within the typical range of heartbeats per minute of an expectant mother, which in a broad range may be from 75 to 95 beats per minute, and in a narrower range from 80 to 90 beats per minute. The 75-95 rate per minute range encompasses virtually all normal expectant mother heartbeat rates, while the 80-90 rate per minute encompasses the majority of normal expectant mother heartbeat rates.

As shown in the illustrations, a representative embodiment of the infant vibratory device comprises an open-topped, cylindrical housing member 11 having a removable cover 12, preferably retained with a threaded coupling. The housing 11 retains a motor 13 secured by a bracket 14, a spinner arm 16 mounted onto the rotating axle or shaft 18 of the motor 13, and a weight 17 affixed to the free outer end of the spinner arm 16 which is mounted onto the shaft 18. Preferably the motor 13 is battery powered and the batteries are likewise retained within the housing 11 in a battery compartment 15. Attachment members are provided on either the cover or the housing, the attachment members preferably comprising brackets 21 adapted to receive a flexible strap member 22 with a releasable closure mechanism, such as for example a hook-and-loop fastener. An activation mechanism, such as a push button or switch, is provided on the exterior of the housing 11 or cover 12 to operate the vibratory device. The battery compartment 15 may be accessed by removing the cover 12 or by providing a removable door on the exterior of the housing 11.

In this manner the infant vibratory device may be temporarily affixed to an infant device, the term infant device being used herein to comprise for example any of the following devices: Baby Gear, Furniture, Cribs, Car Seats, Infant Car Seats, Convertible Car Seats, Booster Car Seats, Car Seat Bases, Car Seat Accessories, Strollers, Frame Strollers, Umbrella Strollers, Full Size Strollers, Travel System Strollers, Lightweight Strollers, Jogging Strollers, Double and Triple Strollers, Stroller Accessories, Baby Carriers, Baby Front and Hip Carriers, Baby Sling and Wrap Carriers, Baby Backpack & Frame Carriers, Baby Carrier Accessories, Bouncers, Infant Seats, Gyms and Playmats, Activity Centers, Jumpers, Swings, Walkers, Play-yards, Portable Beds, Napmats, Infant Recliners, Baby care essentials, Infant Positioners, Baby toys, Crib Toys, Stationary Entertainers, High Chairs and Booster Seats, Bedding, Mattresses.

In a preferred embodiment of the infant vibratory device, the weight 17 is mounted to the spinner arm 16 in a manner that allows replacement of the weight 17 with a heavier or lighter weight 17. Changing the weight 17 within the infant vibratory device will result in vibrations or wobbles at either a faster or slower rate. This feature allows the device to be usable on differing infant devices, since for example a crib may require a stronger or faster vibration or wobble than a lightweight stroller. The inter-changeable weights 17 also allow the vibration rate of the infant vibratory device to be customized to a particular infant.

In the most preferred embodiment, the weight 17 and motor 13 are chosen such that the vibrations per minute or wobbles per minute delivered to the infant device are within the typical range of the heartbeats per minute of an expectant mother, as an infant will be more readily calmed with a vibration rate that equals the mother's heartbeat rate while the infant was in utero. Data shows that the majority of expectant mother heartbeats per minute fall within the range of approximately 80-90 beats per minute, while the vast majority of heartbeats per minute fall within the range of 75-95 beats per minute.

The infant vibratory device is either pre-manufactured at a set vibration rate per minute of between approximately 75-95 vibrations per minute, or more preferably at the rate of between approximately 80-90 vibrations per minute. Alternatively, the infant vibratory device may be manufactured to be adjustable such that the consumer sets the desired vibration rate per minute between 75-95 vibrations per minute. In one method, the proper vibrations per minute rate is accomplished by replacing a weight 17 with a heavier or lighter weight 17. Alternatively, the infant vibratory device may be provided with an external rate adjustment control mechanism, such as for example circuitry to control the speed of the motor 13. Optimally, the consumer will have ascertained her normal heartbeat per minute rate from her physician, and can then set the infant vibratory device to match the precise beats per minute. Before or after setting the device for the proper rate, the infant vibratory device is attached to the infant device. The infant vibratory device is then activated to transfer the vibrations to the infant device to calm the infant.

It is understood that equivalents and substitutions for certain elements described above may be obvious to those of skill in the art, and therefore the true scope and definition of the invention is to be as set forth in the following claims.

I claim:

1. A method of calming an infant retained within an infant device comprising the steps of:
providing an infant vibratory device adapted to produce vibrations at a predetermined vibrations per minute rate, said predetermined vibrations per minute rate being equivalent to the normal heartbeats per minute rate of an expectant mother;
attaching said infant vibratory device to an infant device; and
activating said infant vibratory device to transfer vibrations to said infant device;
wherein said predetermined vibrations per minute rate of said infant vibratory device is within the range of approximately 75 to 95 vibrations per minute.

2. The method of claim 1, wherein said predetermined vibrations per minute rate of said infant vibratory device is within the range of approximately 80 to 90 vibrations per minute.

3. The method of claim 1, wherein said vibrations per minute rate of said infant vibratory device is adjustable, and further comprising the step of adjusting the vibrations per minute rate.

4. The method of claim 2, wherein said vibrations per minute rate of said infant vibratory device is adjustable, and further comprising the step of adjusting the vibrations per minute rate.

5. The method of claim 3, wherein said infant vibratory device is provided with interchangeable weights, and wherein said step of adjusting said infant vibratory device comprises replacing one said weight with a heavier or lighter weight.

6. The method of claim 4, wherein said infant vibratory device is provided with interchangeable weights, and wherein said step of adjusting said infant vibratory device comprises replacing one said weight with a heavier or lighter weight.

7. The method of claim 3, wherein said infant vibratory device comprises a motor, and wherein said step of adjusting said infant vibratory device comprises adjusting the speed of the motor.

8. The method of claim 4, wherein said infant vibratory device comprises a motor, and wherein said step of adjusting said infant vibratory device comprises adjusting the speed of the motor.
